(19) United States
(12) Patent Application Publication
(10) Pub. No.: US 2003/0068306 A1
(43) Pub. Date: Apr. 10, 2003

(54) Medium

(76) Inventor: Mehmet Sirac Dilber, Huddinge (SE)

Correspondence Address: BIRCH STEWART KOLASCH & BIRCH, PO BOX 747, FALLS CHURCH, VA, 22040-0747, US

(21) Appl. No.: 10/242,788

(22) Filed: Sep. 13, 2002

Related U.S. Application Data
(60) Provisional application No. 60/318,871, filed on Sep. 14, 2001.

(51) Int. Cl. A61K 45/00; A61K 39/395; C12N 5/08; A61K 38/20

(52) U.S. Cl. 424/93.7; 424/85.2; 424/144.1; 435/372

(57) ABSTRACT

The invention refers to a medium for expanding natural killer cells expressing the CD56⁺CD3⁻ phenotype, comprising CellGro® SCGM to which has been added interleukin-2, anti CD3 antibodies, and optionally serum, as well as a method of expanding said natural killer cells, isolated from a mononuclear cell concentrate, by suspension and incubation in said medium. The expanded NK cells can be used for curative or prophylactic treatment of patients, especially cancer patients undergoing stem cell transplantation.

This application claims priority under 35 USC 119(e) on provisional application No. 60/318,871 filed (in English) on Sep. 14, 2001, the entire contents of which are incorporated by reference.

The present invention refers to a new medium, which can be used for expanding natural killer cells, and to a method for expanding said cells.

### BACKGROUND OF THE INVENTION

Natural killer cells, NK cells, are defined as cytotoxic cells that have the predominant morphology of large granular lymphocytes and that do not express the CD3 surface antigen complex or any of the known T-cell receptor chains (α, β, γ, δ). In addition the NK cells generally express CD16 and CD56 antigens in humans and the NK1.1 antigen in mouse.

For over twenty years, attempts have been made to cure cancer with adoptive cellular immunotherapy. A major challenge to the successful application of this treatment, for human cancer, has been the identification and expansion of appropriate effector cells. Lymphokine-activated killer (LAK) cells, derived from peripheral blood mononuclear cells (PBMCs) cultured with IL-2, have been characterised extensively in studies of mice and humans [Phillips J H, Lanier L L: Dissection of the lymphokine-activated killer phenomenon. Relative contribution of peripheral blood natural killer cells and T lymphocytes to cytolysis. Journal of Experimental Medicine 164:814, 1986] and are known to lyse a variety of tumour cells through a non-major histocompatibility complex (non-MHC)-restricted mechanism. LAK cells represent a heterogeneous population, in which the major effector cells are NK cells expressing CD56 and CD16 but not CD3. Yet, the therapeutic effectiveness of adoptively transferred LAK cells has, in many cases, been hampered by the cells' inherently low anti-tumour activity in vivo and the difficulty of generating them in large numbers. However, methods for expanding effector-cell populations and increasing their cytotoxic capacity have been improved. So-called CIK (cytokine-induced killer) cells, mainly CD3-positive T-cells approximately 30% of which co-express CD56, have been claimed to possess superior anti-tumour effects in vitro and in human lymphoma-to-SCID (severe combined immunodeficiency)-mouse models compared to LAK cells [Lu P H, Negrin R S: A novel population of expanded human CD3+CD56+ cells derived from T cells with potent in vivo antitumor activity in mice with severe combined immuno-deficiency. Journal of Immunology 153:1687, 1994]. However, when comparing cytotoxicity of sorted subsets of CIK cells on a per-cell basis, the CD3⁻CD56⁺ cells were significantly more potent killers than the CD3⁺CD56⁺ cells [Scheffold C, Brandt K, Johnston V, Lefterova P, Degen B, Schontube M, Huhn D, Neubauer A, Schmidt-Wolf I G: Potential of autologous immunologic effector cells for bone marrow purging in patients with chronic myeloid leukemia. Bone Marrow Transplant 15:33, 1995]. Thus, the NK cells within the LAK- and CIK-cell populations retain the most effective anti-tumour effects. NK cells have attracted further attention in the setting of allogeneic hematopoietic stem cell transplantation (HSCT) and donor leukocyte infusions (DLI). Studies using SCID mice as donors have shown that NK cells transplanted in conjunction with a bone marrow graft and systemic administration of IL-2 promotes superior bone marrow engraftment and mediate anti-tumour effects over that by spleen cells, without inducing graft-versus-host disease (GVHD) [Asai O, Longo D L, Tian Z G, Hornung R L, Taub D D, Ruscetti F W, Murphy W J: Suppression of graft-versus-host disease and amplification of graft-versus-tumor effects by activated natural killer cells after allogeneic bone marrow transplantation. Journal of Clinical Investigation 101:1835, 1998]. When comparing the activity of LAK cells and T cell-depleted LAK cells (mainly NK cells), similar results were obtained. The numbers of circulating NK cells were significantly lower in patients with chronic myelogenous leukaemia (CML) who relapsed-after HSCT than in those who remained in remission, and NK cells were also proposed to be the main effectors of the graft-versus leukaemia (GVL)-effect in the early phase after HSCT [Jiang Y Z, Barrett A J, Goldman J M, Mavroudis D A: Association of natural killer cell immune recovery with a graft-versus-leukemia effect independent of graft-versus-host disease following allogeneic bone marrow transplantation Annals of Hematology 74.1, 1997]. Presumably, then, human CD3⁻CD56⁺ NK cells would be desirable candidates for adoptive transfer it they could be purified and expanded in sufficient amounts.

Other investigators have reported ways of expanding and culturing human NK-cells [Naume B, Gately M, Espevik T: A comparative study of IL-12 (cytotoxic lymphocyte maturation factor)-, IL-2-, and IL-7-induced effects on immunomagnetically purified CD56+ NK cells. J Immunol 148:2429, 1992], although no previous reports have yet dealt with the practical issues of establishing a protocol that potentially could be used for large scale expansion of cells for clinical use.

### SUMMARY OF THE INVENTION

A medium and a protocol for expanding a population of activated human NK cells has been established in which a chemically defined serum-free medium, CellGro® SCGM, in combination with anti CD3 antibodies, IL-2 and optionally serum is used. This cell expansion procedure was optimised to yield 55% CD3⁻CD56⁺ cells, which has been named cytokine-induced natural killer (CINK) cells.

### ABBREVIATIONS

CIK Cytokine induced killer (cells)

CINK Cytokine induced natural killer (cells)

CML Chronic myelogenous leukaemia

DLI Donor leukocyte infusion

FBS Fetal bovine serum

GVHD Graft-versus-host disease

GVL Graft-versus-leukaemia

HS Human serum

HSCT Haematopoietic stem cell transplantation

IL Interleukin

LAK Lymphokine activated killer (cells)

NK Natural killer (cells)

OKT3 Orthoclone, monoclonal antibody against CD3

PBMCs Peripheral blood mononuclear cells

### BRIEF DESCRIPTION OF THE DRAWINGS

FIG. 1. Median cell expansion for seven donors. The cells were cultured in a medium with 500 U/ml IL-2, supplemented with 5% HS, for 5 days and then in a medium without OKT3. Values are presented as median and inter-quartile range.

FIG. 2. The impact of different IL-2 concentrations on the cell expansion in media containing donor PBMCs, OKT3, with and without HS. After 5 days OKT3 was deleted from the media. Data represent median values from three donors, based on medium triplicate wells per combination and donor.

FIG. 3. Absolute numbers of expanded CD3⁻C56⁺ cells (×10⁶), starting with 200,000 cells/well. IL-2 concentrations were raging from 100 to 1000 U/ml in media containing donor PBMCs, OKT3, and with and without HS. After 5 days OKT3 was deleted from the media. Data represent median values from three donors, based on triplicate wells per combination and donor.

FIG. 4. Percent CD3⁻CD56⁺ cells for seven donors. The cells were cultured in media containing 500 U/ml IL-2, supplemented with 5% HS. After 5 days OKT3 was deleted from the media. Values are presented as median and inter-quartile range.

FIG. 5. Evaluation of different serum-free culturing media for the expansion of CD3⁻CD56⁺ cells. The cells were cultured in the following media: CellGro® SCGM, AIM-V, X-VIVO™ 15 and RPMI 1640, to which had been added 500 U/ml IL-2, OKT3 and 5% HS. After 5 days OKT3 was deleted from the media. The data shown represent one of four repeated experiments and are median values from triplicate wells.

FIG. 6. Cytotoxicity of cytokine-induced natural killer (CINK) cells cultured in media with increasing concentrations of IL-2, and supplemented with either HS or FBS. OKT3 was deleted from the media efter 5 days. Cytotoxicity was assessed by a 4-h ⁵¹Cr release assay, with K562 serving as targets at several effector:target ratios.

FIG. 7. The bulk cytotoxic capacity of CINK cells increased over time as the proportion of CD3⁻CD56⁺ cells was increased. The cells were cultured in media with 500 U/ml IL-2, and supplemented with 5% HS. OKT3 was deleted from the media efter 5 days. Cytotoxicity was assessed by a 4-h ⁵¹Cr release assay, with K562 serving as targets at several effector:target ratios.

### DESCRIPTION OF THE INVENTION

The invention refers to a medium for expanding natural killer cells expressing the CD56⁺CD3⁻ phenotype, comprising CellGro® SCGM to which has been added interleukin-2 and anti CD3 antibodies.

The natural killer cells can be obtained from any conventional source, and are preferably derived from peripheral blood, bone marrow, cord blood, cell lines, or cytokine stimulated peripheral blood.

According to a preferred aspect of the invention the medium contains in addition serum, for instance human serum, bovine serum, such as fetal calf serums or horse serum.

The medium of the invention can contain 10-6000 U/ml interleukin-2, 2-50 ng/ml anti CD3 antibodies, 1-40% serum, and optionally additional constituents.

A medium for expanding autologous natural killer cells expressing the CD56⁺CD3⁻ phenotype, should comprise CellGro® SCGM to which has been added interleukin-2, anti CD3 antibodies, and autologous serum.

A medium of the invention could contain CellGro® SCGM to which has been added 50-1000 U/ml interleukin-2, 10-20 ng/ml anti-human CD3 antibodies, and 3-15% human serum. As an example of the composition of a medium can be mentioned that 20 ml of a medium of the invention should contain: 0.2 ml IL-2 (10.000 U), 0.1 ml anti CD3 antibodies (200 ng), 1 ml human serum (5%), 0.2 ml isolated NK cells (2×10⁶ cells), and a remainder of CellGro® SCGM serum free medium.

The medium of the invention can also contain one or more other constituents, such as TNF-alpha, IL-12, IL-1, IK-15, IL-18, interferon alpha/beta, interferon gamma, transferrin, folic acid, or lipopolysaccharides, phytohemagglutinin, ionomycin, and concanavalin.

The invention also refers to a method of expanding natural killer cells expressing the CD56⁺CD3⁻ phenotype, wherein said natural killer cells are isolated from a mononuclear cell concentrate, washed, and then suspended in a medium comprising CellGro® SCGM to which has been added interleukin-2, anti CD3 antibodies, and serum, and incubated in said medium. The NK cells can be concentrated from the mononuclear cell concentrate by density gradient centrifugation or any other conventional process.

To obtain an almost pore population of NK cells, peripheral blood mononuclear cells, PBMN cells, can be separated by density gradient centrifugation, using Ficoll (Nycomed Pharma AS, Oslo, Norway). T cells were depleted from PBMN cells by using anti-CD3 MicroBeads (Miltenyi Biotech, Germany). CD56-positive cells can be separated by using anti-CD56 MicroBeads (Miltenyi Biotech, Germany).

Thus the invention refers to a method of expanding natural killer cells, wherein the natural killer cells have been concentrated by depletion of T cells, and especially to a method of expansion, wherein the natural killer cells have been isolated by positive selection of CD56+ cells and by depletion of T cells (double selection).

The cells to be used in said method can be derived from any suitable source, such as from peripheral blood, bone marrow, cord blood, cell lines, and cytokine stimulated peripheral blood. It has been found that the anti CD3 antibodies can be deleted from the medium after about 3-5 days of incubation without any changes in the expansion of the NK cells, that is a medium without anti CD3 antibodies can be used for the continued cultivation, which should go on for a period of time not less than 5 days, preferably not less than 10 days in order to have a sufficiently pure product in a good yield.

Our findings indicate that large numbers of activated NK cells can now be produced and used in the setting of adoptive immuno-therapy. In leukemia patients autologous expanded NK cells might be helpful for treatment of minimal residual disease (MRD) after autologous stem cell transplantation. It should be possible to administer autologous in vitro cultured, cytokine-induced natural killer cells (CINK) cells, either prophylactically or therapeutically, to patients undergoing autologous hematopoietic stem cell transplantation for diseases such as multiple myeloma which have in general a poor prognosis with high incidence of progressive disease post transplant. In vitro expanded CINK cells of donor origin can be used for the treatment of recurrent malignant disease following allogeneic stem cell transplantation. Autologous CINK cells can be administered, either prophylactically or therapeutically, to patients undergoing autologous stem cell transplantation for cancer. Other ways of using autologous NK cells is for ex viva purging of malignant cells in the harvest, for preventing severe infections after allogeneic or autologous stem cell transplantation, for treatment of patients with hematological malignancies, recurrent or acute infections, patients with allergic or autoimmune diseases, immunodeficient patients, and as a cellular therapy for solid tumours. Another object of the invention is therefore a method of curative or prophylactic treatment, wherein natural killer cells which have been expanded according to the invention are administered to patients with recurrent malignant disease following allogeneic stem cell transplantation, or patients undergoing autologous stem cell transplantation for cancer, or patients with severe infections after allogeneic or autologous stem cell transplantation, or patients with hematological malignancies, recurrent or acute infections or patients with allergic or autoimmune diseases, immunodeficiency, or patients with solid tumours, in a pharmaceutically effective dose

### EXPERIMENTAL

Cell Culture Media and Reagents

Cellgro® SCGM serum-free medium was purchased from CellGenix Technologie Transfer GmbH, Freiburg, Germany

Human serum, HS, from Sigma, St. Louis, Mo.

Fetal bovine serum, FBS, from Gibco, Grand Island, N.Y.

AIM-V, lymphocyte culture medium, from Gibco, Grand Island, N.Y., USA

X-VIVO™ 15, a cell culture medium for tumor infiltrating lymphocytes (TIL), from Biowhittaker, Walksville, Md., USA

RPMI 1640, a basic cell culture medium; from Gibco, Paisley, UK

Interleukin 2, IL-2, with a minimum concentration of 1×10⁷ U/mg, was purchased from Peprotech (London, UK).

Murine anti-human CD3 antibodies, Orthoclone OKT3 were manufactured by Ortho Biotech Inc., Raritan, N.J.

### EXAMPLE 1

### Expansion of NK Cells

Buffy-coat cells were obtained from seven healthy blood-bank donors on the day before starting the cultures. On day 0, Peripheral Blood Mononuclear Cells (PBMC) were isolated by density gradient centrifugation, using Ficoll (Nycomed Pharma AS, Oslo, Norway). The cells were then washed in PBS, their viability assessed by trypan blue dye exclusion, and then the cells (10⁵ cells/ml) were resuspended in the a medium consisting of CellGro® medium supplemented to a final concentration of 10 ng/ml murine anti-human CD3 antibody, OKT3, 500 U/ml recombinant IL-2, 5% (v/v) human serum or 10% (v/v) fetal calf serum, FCS. The complete medium was then plated onto six-well dishes (Falcon by Becton-Dickinson, Meytan Cedex, France) at 2 ml/well. The cells were cultured for 5-6 days. On day 5-6, the cells were washed in PBS and then resuspended in fresh complete medium without OKT3. After this period, complete medium without OKT3 was added regularly throughout the culturing period. On day 10-11 the cells were transferred into T25 flasks (TPP, Trasadingen, Switzerland). Absolute cell numbers were assessed by the cell Coulter technique (Coulter Multisizer II, Coulter Electronics Ltd., Luton, UK) on days 5-6, 10-11 and 21. Viability was analysed with the Trypan blue exclusion assay at each time point. Analyses of lymphocytes, subsets and activation molecules were performed with flow cytometric phenotypic analysis by FACS. Cell mediated cytotoxicity was analysed on NK sensitive cell line, K562 (ATCC, Rockville, Md.), by ⁵¹Cr-release assay [14]. Cultivation in a medium containing OKT3 for 21 days did not result in any statistically different expansion rates, as compared to cultivation in said medium for 5 days and then in a medium without OKT3 for the rest of the time.

In addition, cells from the last three donors were cultured in the complete medium with different concentrations of IL-2 (100 U/ml and 1000 U/ml) as well. The culture conditions were exactly the same as above with exception of IL-2 concentrations.

Analyses of Lymphocyte Subsets and Activation Molecules

Some cultures were prepared for flow cytometric phenotypic analysis. Analysis of three-colour fluorescence was performed according to standard procedures. In short, 10⁵ cells/tube were mixed with appropriate concentrations of fluorochrome-conjugated monoclonal antibodies to CD45/14, T-cell antigens (CD3, CD4, CD8), and to NK-cell antigens (CD56, CD16). All antibodies were obtained from Becton-Dickinson (Becton-Dickinson, Mountain View, Calif.). After the addition of the primary antibody and incubation for 15 minutes at room temperature, cells were washed in PBS, pending analysis. Propidium iodide (PI) staining was used for viability analysis. For data acquisition and analysis, a FACScan (Becton-Dickinson) was used with Cellquest software (Becton-Dickinson). In each sample, 3000 cells were acquired in the analysis region of viable cells, using log-amplified fluorescence and linearly amplified side- and forward-scatter signals.

All samples were analysed by setting appropriate SSC/FSC gates around the lymphocyte population, using back-gating on CD45⁺CD14⁻, PI-negative cells. Consistency of analysis parameters was ascertained by calibrating the flow cytometer with Calibrite beads and the FacsComp software, both from Becton-Dickinson.

Cell Mediated Cytotoxicity

Cells from three donors were analysed on days 0, 5, 10 and 21 of culture, and the NK-sensitive K562 cell line was used as target. Lysis of the cultured cells was measured in a standard 4-hour ⁵¹Cr-release assay using Na₂⁵¹CrO₄-labeled cells in triplicate at various E:T ratios [14]. All cytotoxicity tests were performed using bulk samples of the cultured cells, i.e. no cell sorting was done. The percentage specific ⁵¹Cr release was calculated according to the formula: % release=((experimental release-spontaneous release)/(maximum release-spontaneous release))×100.

Cell Expansion Rates

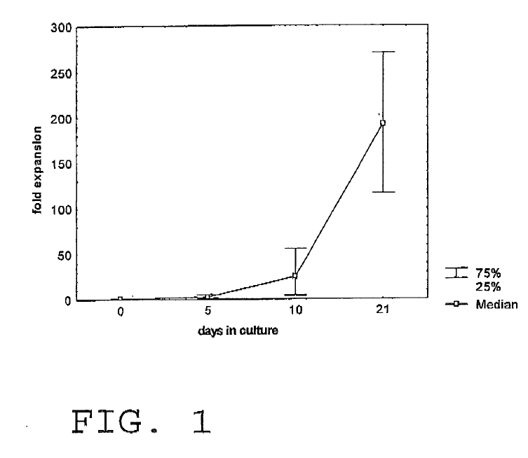

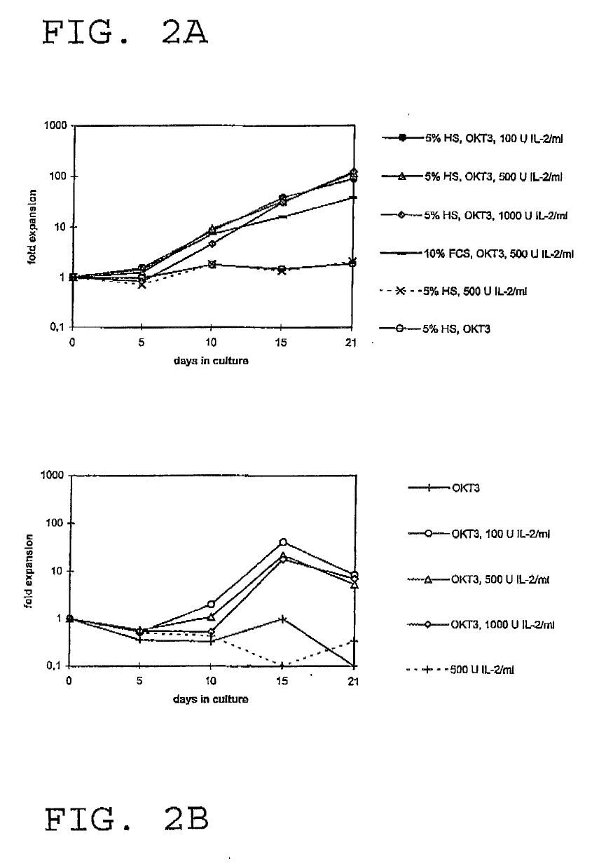

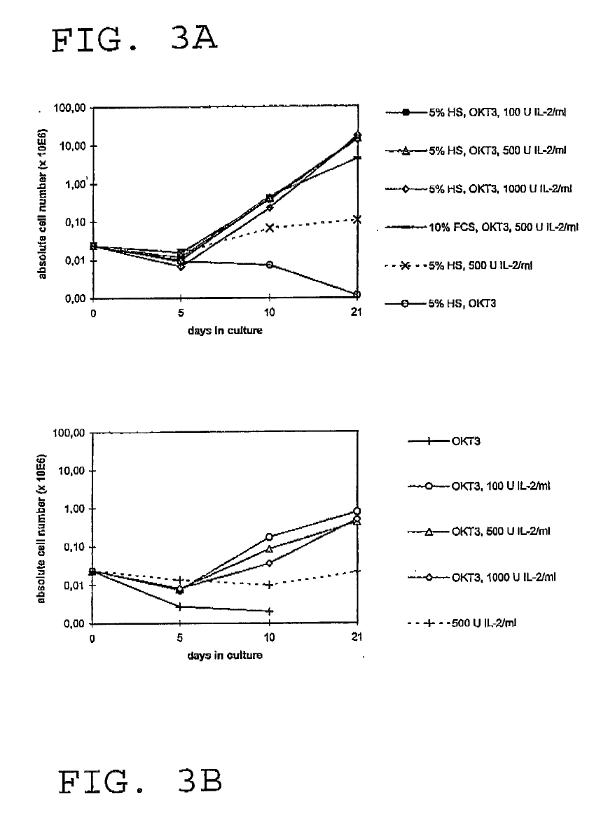

PBMCs originating from seven human donors expanded from the starting number to a median of 193-fold (range 21-277) after 21 days of culture in OKT3 and 5% HS (FIG. 1). Different HS/IL-2/OKT-3 combinations were then used in cultures from three of these donors, and as FIG. 2 depicts, both OKT3 and IL-2 were crucial for the cell expansion process. Cultures lacking either OKT3 or IL-2 completely failed to support cell expansion and, therefore, are not described further. When the HS-supplemented cultures were combined with concentrations of IL-2 varying from 100 to 500 and then to 1000 U IL-2/ml, no major differences in the overall median cell expansion rates were apparent. That is, these cultures expanded to median values of 91-, 116- and 124-fold, respectively. For serum-free cultures the corresponding cell expansions were 8-, 5- and 7-fold As to the absolute number of expanded CD3⁻CD56⁺ cells, the median cell yield (when starting with a total of 0.2×10⁶ cells) was 14, 14 and 17×10⁶ cells for cultures containing 5% HS and 100, 500 and 1000 U IL-2/ml, respectively (FIG. 3). None of the other tested media (AIM-V, X-VIVO 15™ and RPMI 1640) supported the expansion of the CD3⁻CD56⁺ cell subset.

Phenotype of CINK Cells

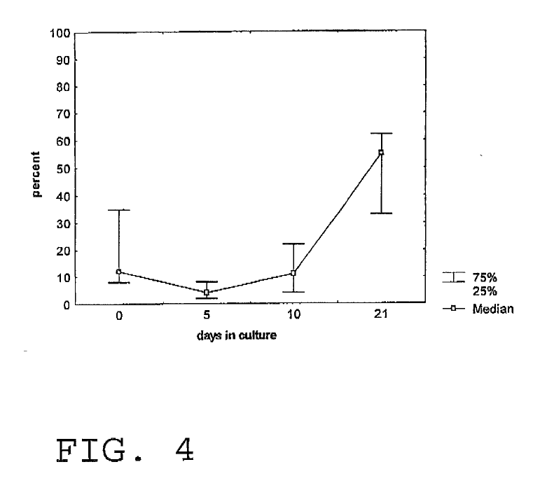

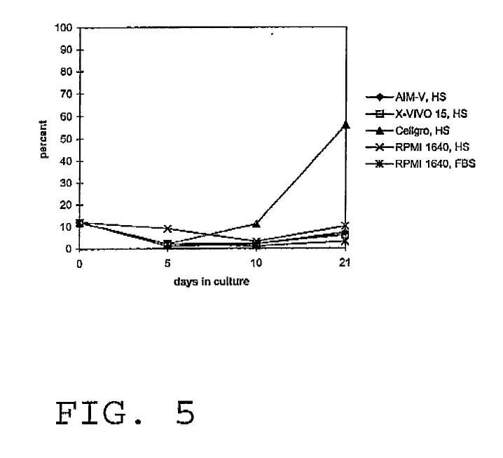

Cells from seven donors, expanded in OKT-3, IL-2 500 U/ml and 5% HS, resulted in a median CINK cell proportion of 55% (range 7-92) CD3⁻CD56⁺ cells (FIG. 4). This selective expansion of NK cells did not occur with any other serum-free media tested here (FIG. 5). A notable inter-donor difference was that cells from donors with low starting numbers of CD3⁻CD56⁺ cells, tended to yield lower final proportions of CD3⁻CD56⁺ cells, than cells from donors with correspondingly higher starting numbers. For example, an initial 5% CD3⁻CD56⁺ cells from one donor reached a final proportion of 7% at the end of the culturing period, in spite of a total 125-fold cell expansion. Another donors sample with as much as 38% CD3⁻CD56⁺ cells in the starting buffy-coat expanded 277-fold, and 92% of these cells were CD3⁻CD56⁺. A median co-expression of the CD16 marker was seen in 78% (range 42-100) of the cultures. Among the CD3⁺ cells (45%) the median value for co-expression of CD56 was 22% (range 2-68); thus, these cells were clearly CIK cells. Twenty percent of total cells were CD3⁺CD4⁺, whereas a median of 5% of the cells co-expressed CD3 and CD8.

Cytotoxic Capacity

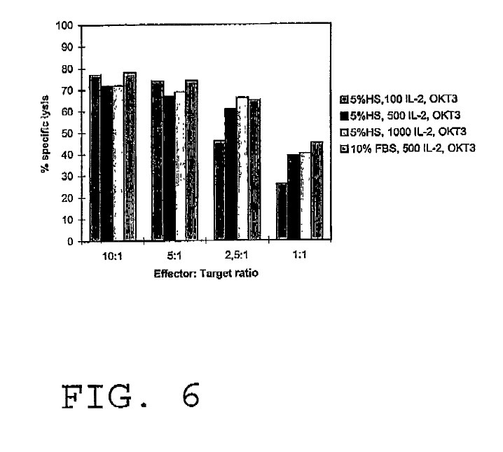

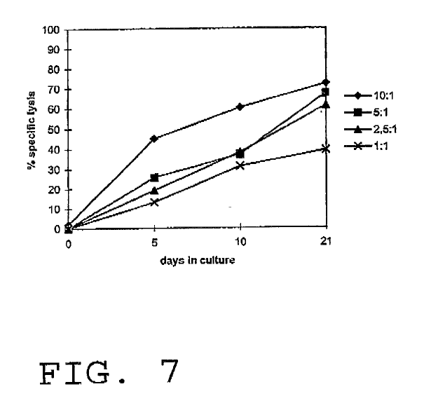

Bulk CINK cells tested in the ⁵¹Cr-release assay showed substantial cytotoxic capacity. The specific release for the 1:1 effector to target ratio ranged from 26 to 45% (FIG. 6). This capacity was not significantly affected by any of the IL-2 concentrations used, nor did the absence of HS or FBS alter the cytotoxicity (data not shown). The total cytotoxic capacity increased over time as the proportion of CD3⁻CD56⁺ cells gradually increased (FIG. 7). However, when the values of specific lysis were adjusted to the actual proportion of CD3⁻CD56⁺ cells in the cultures, the per-cell cytotoxic capacity peaked within the first 10 days of culture, then gradually decreased to the end of the culturing period.

In order to increase the percentage of CINK cells in the final product, T cells have been depleted by using anti-CD3 MicroBeads (Miltenyi Biotech, Germany) from the end product. More than 95% of pure NK cells were obtained after T cell depletion.

### EXAMPLE 2

### Expansion of Enriched NK Cells By Immunomagnetic Beads

Buffy-coat cells were obtained from two healthy blood-bank donors on the day before starting the cultures. On day 0, Peripheral blood mononuclear cells (PBMC) were isolated by density gradient centrifugation, using Ficoll (Nycomed Pharma AS, Oslo, Norway) and then NK cells were separated from PBMN cells by using NE Cell Isolation Kit (Miltenyi Biotech, Germany) with a magnetic labelling system. The NK Cell Isolation Kit is an indirect magnetic labeling system for the isolation of untouched NK cells from peripheral blood. T cells, B cells and myeloid cells are labeled by using a cocktail of hapten-conjugated CD3, CD14, CD19, CD36 and anti-IgE antibodies. The non-NK cells are then magnetically labeled by using MACS MicroBeads coupled to an anti-hapten antibody. Highly pure NK cells with excellent recovery are isolated by retaining the non-NK cells on a column

Percentage of NK cells was round 90% after separation. NK-enriched and non-separated cells were cultured as described in the Example 1 with exception of a cell number of 10⁵/ml in the complete medium. Our complete medium has sufficiently supported NK cells which we were able to obtain almost pure cytokine-induced natural killer, CINK, cell population with more than 50 times expansion after 19 days of culture.

The percentage and number, respectively, of different cells before and after expansion after incubation for up to 19 days are given in the following Table 1.

TABLE 1
Percentage of NK (CD3− CD56+) and other cell types (CD3+ CD56+;
NK-like T cells, CD3+ CD56−; T cells) in the non-separated and NK-
enriched cells at different times. NA = not applicable

| | | Before | After | | | | |
|---|---|---|---|---|---|---|---|
| | | Separa- | Separa- | Day | Day | Day | Day |
| | Day 0 | tion | tion | 6 | 12 | 15 | 19 |
| | CD3+ CD56+ (%) | | | | | | |
| Non- | 11 | NA | NA | 4 | 4 | 8 | 29 |
| separated | | | | | | | |
| cells | | | | | | | |
| Separated | NA | 11 | 83 | 84 | 99 | 97 | 95 |
| cells | | | | | | | |
| | CD3+ CD56+ (%) | | | | | | |
| Non- | 10 | — | — | 12 | 20 | 17 | 21 |
| separated | | | | | | | |
| cells | | | | | | | |
| Separated | — | 10 | 0 | 5 | 1 | 2 | 4 |
| cells | | | | | | | |
| | CD3+ CD56− (%) | | | | | | |
| Non- | 50 | NA | NA | 83 | 74 | 75 | 49 |
| separated | | | | | | | |
| cells | | | | | | | |
| Separated | — | 50 | 5 | 5 | 0 | 0 | 0 |
| cells | | | | | | | |

Number of expanded cells for non-separated and NK-enriched cells.

| | Cell Expansion (10⁶) | | | | |
|---|---|---|---|---|---|
| | Day 0 | Day 6 | Day 12 | Day 15 | Day 19 |
| Non- | 0.2 | 5 | 16 | 35 | 78 |
| separated | | | | | |
| cells | | | | | |
| Separated | 0.2 | 1 | 10 | 29 | 56 |
| cells | | | | | |

### EXAMPLE 3

### Expansion of Autologous NK Cells

The opportuneness of using autologous CINK cells in hematological malignancies, in particular lymphomas has been explored. The feasibility of expansion of CINK cell have been tested by the method described in Example 1. The cytotoxic effect of in vitro expanded CINK cells have also been evaluated against different tumor cell lines and their autologous tumor cells at day 20.

Preliminary data has shown an expansion of CINK cells in 4/4 tested B cell lymphocytic leukemia, B-CLL, patients. Moreover, also after fludarabine treatment an expanded CINK-cell population was seen in 2/2 patients. Data from two representative patients are demonstrated in Table 2.

TABLE 2
Percentage of NK (CD3− CD56+) and other cell types (CD19+; Tumor
and normal B-cells, CD3+ CD56+; NK-like T cells, CD3+ CD56−; T
cells) at different culture periods. Cells were obtained from
peripheral blood mononuclear cells of patients with B-type chronic
lymphocytic leukemia (B-CLL) at Day 0.

| | Day 0 | Day 5-6 | Day 9-11 | Day 14-15 | Day 19-21 |
|---|---|---|---|---|---|
| | CD19+ (%) | | | | |
| Patient 1 | 62 | 19 | 9 | ND | 1 |
| Patient 2 | 41 | 3 | 1 | 1 | 0 |
| | CD3− CD56+ (%) | | | | |
| Patient 1 | 4 | 7 | 22 | ND | 87 |
| Patient 2 | 11 | 15 | 20 | 41 | 54 |
| | CD3+ CD56− (%) | | | | |
| Patient 1 | 7 | 66 | 55 | ND | 10 |
| Patient 2 | 39 | 73 | 63 | 45 | 30 |
| | CD3+ CD56+ (%) | | | | |
| Patient 1 | 0 | 3 | 8 | ND | 2 |
| Patient 2 | 3 | 3 | 16 | 12 | 15 |

Number of expanded total cells during culture period. ND: Not
determined

| | Cell expansion (10⁶) | | | | |
|---|---|---|---|---|---|
| Patient 1 | 2 | 9 | 12 | ND | 59 |
| Patient 2 | 1 | 3 | 10 | 31 | 164 |

### CLINICAL TRIAL

Infusion of Donor Derived CINK For the Treatment of Recurrent Malignant Disease After Allogeneic Hematopoietic Stem Cell Transplantation

A non-randomized phase I-II pilot trial evaluating the safety and toxicity of adaptively transferred donor CINK cells is performed. Candidates to be treated are patients with progressive malignant disease following allogeneic hematopoietic stem cell transplan-tation for one of the following diagnoses: CML, AML, ALL, hepato-cellular carcinoma, colon carcinoma, prostate cancer, renal carcinoma or other cancer. They should have an HLA -A, -B, -DR identical, related stem cell donor (sibling or parent) who is eligible for, and willing to undergo a leukapheresis. They should also have signs of progressive disease, by MRD (minimal-residual disease by PCR), mixed chimerism and increasing levels of tumor antigens. Stem cell donors will undergo unstimulated peripheral lymphapheresis on one or two occasions, depending on the number of cells retrieved, Cells are then activated according to the specific protocol of the invention and cultured for 21 days in a so called closed-culture system for optimal sterility and reproducibility. The ready expanded cell culture is tested for sterility, viability and phenotypic expression at the end of the culturing period. Expanded cells that are not immediately transferred to the recipient are cryopreserved for later use.

All immunosuppressive treatment must be stopped and no signs of GVHD should be present prior to the infusion of the CINK cells. Also, signs of progressive disease must remain unaffected by the discontinuation of any immunosuppressive treatment. One month after discontinuing the immunosuppressive treatment the first DLI dose is given. Prior to each DLI, the phenotype of the CINK cells are confirmed by FACS. The expanded cells are administered through a central venous line in escalating doses 10⁶ to 10⁸ CD56 positive cells/kg bodyweight of the recipient with a month interval for three times.

Patients will be monitored weekly for: detailed clinical history, physical examination and skin evaluation when appropriate, general laboratory evaluation according to existing routines for follow up after allogeneic stem cell transplantation. Laboratory evaluation of NK-cell chimerism a total NK-cell number after each cell infusion. Disease status will be monitored and assessed in accordance to each underlying disease.

### CONCLUSION

This study demonstrates a method for in vitro expansion of CD3⁻CD56⁺ cells, originating from peripheral blood mononuclear cells of human donors. We found that our method enabled cells to expand 193-fold (range 21-277) in 21 days in cultures supplemented with 5% human serum and IL-2 (500 U/ml). This expanded population, here named CINK cells, comprised 55% (median, range 7-92) CD3⁻CD56⁺ cells. Both IL-2 and OKT-3 proved to be essential for this procedure. Previously, cells with this phenotype mediated more potent cytotoxic effects than any other subset of activated lymphocytes [8-10]. The fact that, to our knowledge, no clinical trials have been performed in which NK cells or activated NK cells are adoptively transferred as immunotherapy is most likely due to the traditional difficulty of culturing and enriching human CD3⁻CD56⁺ cells in large amounts.

In tests of the CD3⁻CD56⁺ cells' cytotoxic capacity, we used as targets the NK-sensitive cell line, K562 The results showed that the expanded cell population prepared by our method lysed 26 to 45% of the target cells in a 1:1 effector to target ratio, signifying substantial cytotoxic efficacy. Also, a large proportion (78%) of the CD3⁻CD56⁺ cells expressed of the CD16 marker by day 21 of culture, indicating a state of activation.

Preliminary experiments comparing the cytotoxicity between pure FACS sorted CD3³¹ CD56⁺ cells and CD3⁻CD56⁺ cells have indicated a 4-6 fold increased cytotoxicity at the 1:1 effector to target ratio for the CD3⁻CD56⁺ cell subset compared to the CD3⁺CD56⁺ cells (data not shown).

The results cited here offer reasons to believe that preparing a CD56 enriched (and predominantly CD3⁻) cell infusion is practical and that its anti-tumour effects will be an improvement over results with naive T-cells, LAK-cells or CIK-cells.

1. Medium for expanding natural killer cells expressing the CD56⁺CD3⁻ phenotype, comprising CellGro® SCGM to which has been added interleukin-2 and anti CD3 antibodies.

2. Medium according to claim 1, wherein the natural killer cells are derived from a member selected from the group consisting of peripheral blood, bone marrow, cord blood, cell lines, cytokine stimulated peripheral blood.

3. Medium according to claim 1, containing in addition serum.

4. Medium according to claim 3, wherein the serum is selected from the group consisting of human serum, bovine serum and horse serum.

5. Medium according to any of claims 1-4, containing 10-6000 U/ml interleukin-2, 2-50 ng/ml anti CD3 antibodies, 1-40% serum, and optionally additional constituents.

6. Medium for expanding autologous natural killer cells expressing the CD56⁺CD3⁻ phenotype, comprising CellGro® SCGM to which has been added interleukin-2, anti CD3 antibodies, and autologous serum.

7. Medium according to claim 1 or 6, consisting of CellGro® SCGM to which has been added 50-1000 U/ml interleukin-2, 10-20 ng/ml anti-human CD3 antibodies, and 3-15% human serum.

8. Medium according to any of claims 1-7, containing one or more of the constituents selected from the group consisting of TNF-alpha, IL-12, IL-1, IL-15, IL-18, interferon alpha/beta, interferon gamma, transferrin, folic acid, lipopolysaccharides, phytohemagglutinin, ionomycin, and concanavalin.

9. Method of expanding natural killer cells expressing the CD56⁺CD3⁻ phenotype, wherein said natural killer cells are isolated from a mononuclear cell concentrate, washed, and then suspended in a medium comprising CellGro® SCGM to which has been added interleukin-2, anti CD3 antibodies, and serum, and incubated in said medium.

10. Method according to claim 9, wherein the natural killer cells have been concentrated by depletion of T cells.

11. Method according to claim 9, wherein the natural killer cells have been isolated by means or a NK cell separation kit.

12. Method according to claim 9, wherein the natural killer cells have been isolated by means of beads coated with anti-CD56 antibodies.

13. Method according to claim 9, wherein the natural killer cells have been isolated by positive selection of CD56+ cells and by depletion of T cells (double selection).

14. Method according to any of claims 9-13, wherein the cells are derived from a member selected from the group consisting of peripheral blood, bone marrow, cord blood, cell lines, cytokine stimulated peripheral blood.

15. Method according to claim 9, wherein the anti CD3 antibodies can be deleted from the medium after about 3-5 days of incubation.

16. Method according to claim 9, wherein the natural killer cells are incubated for a period of time not less than 5 days.

17. Method of curative or prophylactic treatment, wherein natural killer cells which have been expanded according to any of claims 9-16 are administered to patients with recurrent malignant disease following allogeneic stem cell transplantation, or patients undergoing autologous stem cell transplantation for cancer, or patients with severe infections after allogeneic or autologous stem cell transplantation, or patients with hematological malignancies, recurrent or acute infections or patients with allergic or autoimmune diseases, immunodeficiency, or patients with solid tumours, in a pharmaceutically effective dose.
